SEQUENCE LISTING (26 sequences)

SEQ ID NO: 1 - DNA, 20 bp, Artificial Sequence
  primer
agagtttgatcatggctcag

SEQ ID NO: 2 - DNA, 17 bp, Artificial Sequence
  primer
aaggaggtgatccaacc

SEQ ID NO: 3 - DNA, 1542 bp, Serratia ficaria
aaattgaagagtttgatcatggctcagattgaacgctggcggcaggcctaacacatgcaagtcgagcggtagcacaggagagcttgctctctgggtgacgagcggcggacgggtgagtaatgtctgggaaactgcctgatggagggggataactactggaaacggtagctaataccgcataacgtctacggaccaaagtgggggaccttcgggcctcacgccatcagatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaagggcgatgtcttaatacggcatcgcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgcgcttaacgtgggaactgcatttgaaactggcaagctagagtctcgtagaggggggtagaattccaggtgtagcggtgaaatgcgtagagatctggaggaataccggtggcgaaggcggccccctggacgaagactgacgctcaggtgcgaaagcgtggggagcaaacaggattagataccctggtagtccacgctgtaaacgatgtcgatttggaggttgtgcccttgaggcgtggcttccggagctaacgcgttaaatcgaccgcctggggagtacggccgcaaggttaaaactcaaatgaattgacgggggcccgcacaagcggtggagcatgtggtttaattcgatgcaacgcgaagaaccttacctactcttgacatccagagaactttccagagatggattggtgccttcgggaactctgagacaggtgctgcatggctgtcgtcagctcgtgttgtgaaatgttgggttaagtcccgcaacgagcgcaacccttatcctttgttgccagcgattcggtcgggaactcaaaggagactgccggtgataaaccggaggaaggtggggatgacgtcaagtcatcatggcccttacgagtagggctacacacgtgctacaatggcgtatacaaagagaagcgaactcgcgagagcaagcggacctcataaagtacgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 4 - DNA, 1542 bp, Ralstonia pikettii; identical to SEQ ID NO: 3

SEQ ID NO: 5 - DNA, 1531 bp, Caldimonas manganoxidans
aaattgaagagtttgatcatggctcagattgaacgctggcggcatgctttacacatgcaagtcgaacggcagcgggtccttcgggatgccggcgagtggcgaacgggtgagtaatgcatcggaacgtacccagtcgtgggggataactactcgaaagagtagctaataccgcatacgacctgagggtgaaagcgggggaccgcaaggcctcgcgcgattggagcggccgatgtcggattagctagttggtggggtaaaggcttaccaaggcgacgatccgtagctggtctgagaggacggccagccacactgggactgagacacggcccagactcctacgggaggcagcagtggggaattttggacaatgggcgcaagcctgatccagccatgccgcgtgcgggaagaaggccttcgggttgtaaaccgcttttgtcagggaagaaaagctctgggctaatacctcggagtgatgacggtacctgaagaataagcaccggctaactacgtgccagcagccgcggtaatacgtagggtgcgagcgttaatcggaattactgggcgtaaagcgtgcgcaggcggttgtgcaagacagatgtgaaatccccgggcttaacctgggaactgcatttgtgactgcacggctagagtgcggcagagggggatggaattccgcgtgtagcagtgaaatgcgtagatatgcggaggaacaccgatggcgaaggcagtcccctgggcctgcactgacgctcatgcacgaaagcgtggggagcaaacaggattagataccctggtagtccacgccctaaacgatgtcaactggttgttggggattcatttcctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacctacccttgacatgccaggaaccctgcagagatgtgggggtgctcgaaagagagcctggacacaggtgctgcatggccgtcgtcagctcgtgtcgtgagatgttgggttaagtcccgcaacgagcgcaacccttgccattagttgctacgaaagggcactctaatgggactgccggtgacaaaccggaggaaggtggggatgacgtcaggtcctcatggcccttatgggtagggctacacacgtcatacaatggccggtacagagggctgccaacccgcgagggggagccaatcccagaaaaccggtcgtagtccggatcgcagtctgcaactcgactgcgtgaagtcggaatcgctagtaatcgcggatcagcatgtcgcggtgaatacgttcccgggtcttgtacacaccgcccgtcacaccatgggagcgggttctgccagaagtgggtagcctaaccgcaaggagggcgcttaccacggcagggttcgtgactggggtgaagtcgtaacaaggtaaccgtaggggaacctgcggttggatcacctcctta

SEQ ID NO: 6 - DNA, 1529 bp, Hydrogenophaga flava
aaattgaagagtttgatcatggctcagattgaacgctggcggcatgctttacacatgcaagtcgaacggtaacaggccgcaaggtgctgacgagtggcgaacgggtgagtaatgcatcggaacgtgcccagtcgtgggggataacgcagcgaaagctgtgctaataccgcatacgatctatggatgaaagcgggggaccgtaaggcctcgcgcgattggagcggccgatgtcagattaggtagttggtggggtaaaggctcaccaagccaacgatctgtagctggtctgagaggacgaccagccacactgggactgagacacggcccagactcctacgggaggcagcagtggggaattttggacaatgggcgcaagcctgatccagcaatgccgcgtgcaggaagaaggccttcgggttgtaaactgcttttgtacggaacgaaacggtctgggttaatacctcgggctaatgacggtaccgtaagaataagcaccggctaactacgtgccagcagccgcggtaatacgtagggtgcaagcgttaatcggaattactgggcgtaaagcgtgcgcaggcggttttgtaagacaggcgtgaaatccccgggctcaacctgggaattgcgcttgtgactgcaaggctggagtgcggcagagggggatggaattccgcgtgtagcagtgaaatgcgtagatatgcggaggaacaccgatggcgaaggcaatcccctgggcctgcactgacgctcatgcacgaaagcgtggggagcaaacaggattagataccctggtagtccacgccctaaacgatgtcaactggttgttgggaatttactttctcagtaacgaagctaacgcgtgaagttgaccgcctggggagtacggccgcaaggttgaaactcaaaggaattgacggggacccgcacaagcggtggatgatgtggtttaattcgatgcaacgcgaaaaaccttacccacctttgacatggcaggaagtttccagagatggattcgtgctcgaaagagaacctgcacacaggtgctgcatggctgtcgtcagctcgtgtcgtgagatgttgggttaagtcccgcaacgagcgcaacccttgccattagttgctacgaaagggcactctaatgggactgccggtgacaaaccggaggaaggtggggatgacgtcaagtcctcatggcccttataggtggggctacacacgtcatacaatggccggtacaaagggcagccaacccgcgagggggagccaatcccataaagccggtcgtagtccggatcgcagtctgcaactcgactgcgtgaagtcggaatcgctagtaatcgtggatcagcatgtcacggtgaatacgttcccgggtcttgtacacaccgcccgtcacaccatgggagcgggtctcgccagaagtagttagcctaaccgcaaggagggcgattaccacggcggggttcgtgactggggtgaagtcgtaacaaggtaaccgtaggggaacctgcggttggatcacctcctta

SEQ ID NO: 7 - DNA, 1536 bp, Oligella urethralis
aaattgaagagtttgatcctggctcaattgaacgctagcgggatgctttacacatgcaagtcgaacggcagcatgagagagcttgctctcttgatggcgagtggcggacgggtgagtaatgtatcggaacgtgcccggtagtgggggataactacgcgaaagcgtagctaataccgcatattctctacggaggaaagagggggatcgtaagacctcttgctaccggagcggccgatatcagattagctggttggtggggtaacggcctaccaaggcggtgatctgtagctggtttgagaggacgaccagccacactgggactgagacacggcccagactcctacgggaggcagcagtggggaattttcgacaatggggggaaccctgatcgagccatctcgcgtgtgggacgaaggccttcgggttgtaaaccacttttctcaacgaagaaaagtcttaggctaataccttaagatgctgacggtagttgaggaataagcaccggctaactacgtgccagcagccgcggtaatacgtagggtgcgagcgttaatcggaattactgggcgtaaagggtgcgcaggcggcttggaaagaacggtgtgaaatcccggggctcaacctcggaactgcactattaactgccaagctagagtatgtcagaggggggtggaattccgcgtgtagcagtgaaatgcgtagatatgcggaggaacaccgatggcgaaggcagccccctgggatggtactgacgctcaggcacgaaagcgtgggtagcgaacaggattagataccctggtagtccacgccctaaacgatgtcaactagctgttgggccggttacggcttagtagcgcagctaacgcgtgaagttgaccgcctggggactacggtcgcaagactaaaactcaaaggaattgacggggacccgcacaagcggtggatgatgtggattaattcgatgcaacgcgaaaaaccttacctacccttgacatgcccggaagcttcaagagattggagtgtgctcgcaagagaaccggagcacaggtgctgcatggctgtcgtcagctcgtgtcgtgagatgttgggttaagtcccgcaacgagcgcaacccttgtcattagttgctacgaaagggcactctaatgagactgccggtgacaaaccggaggaaggtggggatgacgtcaagtcctcatggcccttatgggtagggcttcacacgtcatacaatggtcaggacagcgggttgccaagccgcgaggtggagctaatctcttaaacctgatcgtagtccggattgcaggctgcaactcgcctgcatgaagtcggaatcgctagtaatcgcagatcagaatgctgcggtgaatacgttcccgggtcttgtacacaccgcccgtcacaccatgggagtgggttttaccagaagtagttagcctaaccgcaaggagggcgattaccacggtaggattcatgactggggtgaagtcgtaacaaggtaaccgtaggggaacctgcggttggatcacctcctta

SEQ ID NO: 8 - DNA, 1537 bp, Oxalicibacterium horti
aaattgaagagtttgatcatggctcagattgaacgctggcggcatgccttacacatgcaagtcgaacggcagcacgggagcttgctcctggtggcgagtggcgaacgggtgagtaatatatcggaacgtacccagtagtgggggatagcccggcgaaagccggattaataccgcatacgatctacggatgaaagcgggggatcgcaagacctcgcgctattggagcggccgatatctgattagctagttggtagggtaaaagcctaccaaggctacgatcagtagctggtctgagaggacgaccagccacactggaactgagacacggtccagactcctacgggaggcagcagtggggaattttggacaatgggcgaaagcctgatccagcaatgccgcgtgagtgaagaaggccttcgggttgtaaagctcttttgtcagggaagaaacggtagaggctaatatcctttgctaatgacggtacctgaagaataagcaccggctaactacgtgccagcagccgcggtaatacgtagggtgcaagcgttaatcggaattactgggcgtaaagcgtgcgcaggcggttgtgcaagacagatgtgaaatccccgggctcaacctgggaattgcatttgtgactgcacggctagagtgtgtcagaggggggtagaattccacgtgtagcagtgaaatgcgtagagatgtggaggaataccgatggcgaaggcagccccctgggataacactgacgctcatgcacgaaagcgtggggagcaaacaggattagataccctggtagtccacgccctaaacgatgtctactagttgtcgggtcttaattgacttggtaacgcagctaacgcgtgaagtagaccgcctggggagtacggtcgcaagattaaaactcaaaggaattgacggggacccgcacaagcggtggatgatgtggattaattcgatgcaacgcgaaaaaccttacctacccttgacatgtacggaagctttgagagatcagagtgtgctcgaaagagagccgtaacacaggtgctgcatggctgtcgtcagctcgtgtcgtgagatgttgggttaagtcccgcaacgagcgcaacccttgtcattagttgctacatttagttgagcactctaatgagactgccggtgacaaaccggaggaaggtggggatgacgtcaagtcctcatggcccttatgggtagggcttcacacgtcatacaatggtacatacagagggccgccaacccgcgagggggagctaatcccagaaagtgtatcgtagtccggattgtagtctgcaactcgactacatgaagttggaatcgctagtaatcgcggatcagcatgtcgcggtgaatacgttcccgggtcttgtacacaccgcccgtcacaccatgggagcgggtttcaccagaagtgggtagcctaaccgcaaggagggcgctcaccacggtgggattcgtgactggggtgaagtcgtaacaaggtaaccgtaggggaacctgcggttggatcacctcctta

SEQ ID NO: 9 - DNA, 1537 bp, Spirillum lunatum
aaattgaagagtttgatcatggctcagattgaacgctggcggcaggcctaacacatgcaagtcgagcggatgaagggagcttgctccctgattcagcggcggacgggtgagtaatgcctaggaatctgcctggtagtgggggacaacgtctcgaaagggacgctaataccgcatacgtcctacgggagaaagcaggggaccttcgggccttgcgctatcagatgagcctaggtcggattagctagttggtgaggtaatggctcaccaaggctgcgatccgtaactggtctgagaggatgatcagtcacactggaactgagacacggtccagactcctacgggaggcagcagtggggaatattggacaatgggcgaaagcctgatccagccatgccgcgtgtgtgaagaaggtcttcggattgtaaagcactttaagttgggaggaagggcagtaagttaataccttgctgttttgacgttaccgacagaataagcaccggctaacttcgtgccagcagccgcggtaatacgaagggtgcaagcgttaatcggaattactgggcgtaaagcgcgcgtaggtggtttgttaagttggaagtgaaagccccgggctcaacctgggaactgctttcaaaactggcaagctagagtatggcagagggtggtggaatttcctgtgtagcggtgaaatgcgtagatataggaaggaacaccagtggcgaaggcgaccacctgggctaatactgacactgaggtgcgaaagcgtggggagcaaacaggattagataccctggtagtccacgccgtaaacgatgtcgactagccgttgggctccttgagagcttagtggcgcagctaacgcattaagtcgaccgcctggggagtacggccgcaaggttaaaactcaaatgaattgacgggggcccgcacaagcggtggagcatgtggtttaattcgaagcaacgcgaagaaccttaccaggccttgacatgcagagaactttccagagatggattggtgccttcgggagctctgacacaggtgctgcatggctgtcgtcagctcgtgtcgtgagatgttgggttaagtcccgtaacgagcgcaacccttgtccttagttaccagcacgttaaggtgggcactctaaggagactgccggtgacaaaccggaggaaggtggggatgacgtcaagtcatcatggcccttacggcctgggctacacacgtgctacaatggtcggtacaaagggttgccaagccgcgaggtggagctaatcccataaaaccgatcgtagtccggatcgcagtctgcaactcgactgcgtgaagtcggaatcgctagtaatcgtgaatcagaatgtcacggtgaatacgttcccgggccttgtacacaccgcccgtcacaccatgggagtgggttgctccagaagtagctagtctaaccttcgggaggacggttaccacggagtgattcatgactggggtgaagtcgtaacaaggtaaccgtaggggaacctgcggttggatcacctcctta

SEQ ID NO: 10 - DNA, 1532 bp, Burkholderia sacchari
aaattgaagagtttgatcatggctcagattgaacgctggcggcatgccttacacatgcaagtcgaacggcagcacgggtgcttgcacctggtggcgagtggcgaacgggtgagtaatacatcggaacatgtccagtagtgggggatagcccggcgaaagccggattaataccgcatacgctctacggaggaaagcgggggatcgcaagacctcgcgctattggggtggccgatggcagattaggtagttggtggggtaaaggcctaccaagccgacgatctgtagctggtctgagaggacgaccagccacactgggactgaaccgggcccacactcctacgggaggcagcagtggggaattttggacaatgggcgcaagcctgatccagcaatgccgcgtgtgtgaagaaggccttcgggttgtaaagcacttttgtccggaaagaaatcctctgggttaataccccggggggatgacggtaccggaagaataagcaccggctaactacgtgccagcagccgcggtaatacgtagggtgcaagcgttaatcggaattactgggcgtaaagcgtgcgcaggcggtgatgcaagaccgatgtgaaatccccgggcttaacctgggaactgcattggtgactgcatcgctagagtatggcagaggggggtagaattccacgtgtagcagtgaaatgcgtagagatgtggaggaataccgatggcgaaggcagccccctgggccaatactgacgctcatgcacgaaagcgtggggagcaaacaggattagataccctggtagtccacgccctaaacgatgtcaactggttgtcgggccttcattggcttggtaacgtagctaacgcgtgaagttgaccgcctggggagtacggtcgcaagattaaaactcaaaggaattgacggggacccgcacaagcggtggatgatgtggattaattcgatgcaacgcgaaaaaccttacctacccttgacatgtacggaagtctgccgagaggtggatgtgcccgaaagggagccgtaacacaggtgctgcatggctgtcgtcagctcgtgtcgtgagatgttgggttaagtcccgcaacgagcgcaacccttgtccccagttgctacgcaagagcactccggggagactgccggtgacaaaccggaggaaggtggggatgacgtcaagtcctcatggcccttatgggtagggcttcacacgtcatacaatggtcggaacagagggttgccaagccgcgaggtggagccaatcccagaaaaccgatcgtagtccggatcgcagtctgcaactcgactgcgtgaagctggaatcgctagtaatcgcggatcagcatgccgcggtgaatacgttcccgggtcttgtacacaccgcccgtcacaccatgggagtgggttttgccagaagtggctagtctaaccgcaaggaggacggtcaccacggcaggattcatgactggggtgaagtcgtaacaaggtaaccgtaggggaacctgcggttggatcacctcctta

SEQ ID NO: 11 - DNA, 714 bp, Agaricus bisporus
atgagcaagggagaggagctgttcaccggcgtcgtgcccatcctcgtggagctggacggcgacgtgaacgggcacaagttctccgtccgtggggagggcgagggcgacgccacaaacgggaagctgacactgaagtttatctgcacgaccggcaagctgcctgtgccatggccgaccctggtcaccacgctgacctacggcgtgcagtgcttcagcaggtatcccgaccatatgaagcgtcacgactttttcaagagcgccatgcccgagggctacgtgcaagagcgtaccatcagctttaaggacgatggcacctacaagacgcgagcggaggtgaagttcgagggagacaccctggtgaaccgtatcgagctcaaggggatcgacttcaaggaggacggcaacatcctgggccacaaactcgagtacaacttcaacagccacaacgtgtacatcaccgcagataagcagaaaaacggcatcaaggccaacttcaaaatccggcacaatgtggaggacggaagcgtgcagctggccgatcactaccagcagaacacccctatcggcgacggaccagtgctgctgcccgacaatcactacctgagcacgcagtccgtgctgagcaaagaccccaacgaaaagcgcgaccacatggtgctgctggagttcgtgaccgctgccggcatcacccacggcatggacgaactgtacaag

SEQ ID NO: 12 - DNA, 714 bp, Bacillus subtilis
atgtctaagggagaagagcttttcacaggtgtcgttcctatactggtggagttagacggcgacgtgaatggtcacaagtttagcgttagaggtgaaggcgagggcgatgcgaccaatggaaaactcacacttaagtttatctgcactaccggaaaactcccagtgccgtggcctacgctggtaactacacttacctatggcgtccaatgttttagccgatatccggatcacatgaaacgtcatgactttttcaaatcagccatgccggaagggtatgttcaggaacgcacgatttcattcaaggatgatggaacatacaaaacgcgcgcagaagtcaaattcgaaggtgatacattggtaaatcggattgaactgaaagggattgacttcaaagaggatggcaatatcctggggcataagttagaatacaacttcaattcgcataacgtctacattactgctgataaacagaaaaacggaatcaaagccaacttcaaaatcagacataatgtcgaagatgggagcgttcaattagctgatcattatcaacaaaacacgccgattggcgatggaccagtgttgttaccggataatcattacttgtccacacagtcagttcttagtaaagatcctaacgagaagcgtgaccacatggtgttgcttgaatttgtaacggcggcaggcattacacatggaatggacgaactgtacaaa

SEQ ID NO: 13 - DNA, 714 bp, Escherichia coli
atgagcaaaggcgaagaactgttcaccggcgtcgtcccaatcctggttgagttggacggtgacgtcaacggtcacaagtttagcgtgcgtggcgaaggcgagggcgacgcgaccaatggcaagttgacgctgaaattcatttgcaccaccggtaagctgccggttccgtggccgactctggttacgacgctgacctacggcgtgcagtgtttctctcgttacccggatcacatgaaacgccacgatttcttcaagagcgcgatgccggaaggttatgttcaggagcgcacgattagctttaaagacgatggtacctacaaaacccgtgctgaggtgaagtttgaaggtgacaccctggtcaaccgtattgagctgaaaggtatcgatttcaaagaggacggtaacattctgggccataaactggaatacaatttcaatagccacaacgtctatatcaccgcggacaagcagaagaacggtatcaaagcaaactttaagatccgccataacgttgaggatggctcggttcaattggcggaccattaccagcaaaatacgccgatcggtgacggtccggtattgctgcctgataatcattatctgtccactcaaagcgtgctgagcaaagatccgaatgagaagcgtgaccacatggtgctgctggagtttgtgacggcagccggtattacccacggtatggatgaactgtataag

SEQ ID NO: 14 - DNA, 714 bp, Homo sapiens
atgtccaagggagaagaattgttcacgggagtcgtacctatcctcgtagaacttgatggcgatgtcaacggacacaagttctcggtgcgcggtgagggggagggagatgccacgaatgggaaattgaccctgaagtttatctgcactacgggaaagctgccggtcccttggccgactctcgtgactacgttgacgtacggggtacaatgttttagccgctatcccgaccatatgaaaagacatgactttttcaaaagcgcgatgcccgaggggtatgtgcaagaacgaacgatctcgtttaaggacgacggcacatacaagacaagagccgaagtgaagttcgagggagatacactggtcaaccggatcgagctcaagggtattgacttcaaagaggatggaaacattctcgggcacaaattggagtacaatttcaactcacataatgtctatatcacagctgacaagcagaaaaacggcattaaggcgaatttcaaaatccggcacaatgtagaagatggatcggtgcagctcgcggatcactatcagcagaacacacccatcggtgacggaccagtcttgctccccgataaccactacctttccacccagagcgtcctgtcaaaagacccgaatgagaagagggatcacatggtacttcttgagtttgtgactgccgcagggattacccatgggatggacgaactttacaag

SEQ ID NO: 15 - DNA, 714 bp, Saccharomyces cerevisiae
atgtcaaaaggtgaggaattgtttacaggcgttgtccctattctagttgaattggatggtgacgtcaatggtcataagttttccgttagaggcgaaggtgaaggtgatgctaccaacgggaagttaactcttaagtttatctgtacaactggaaagctaccagttccttggccaacactggttacaactttaacttacggcgtccaatgctttagtagatacccagaccatatgaaacgtcatgactttttcaaatctgcaatgccagagggttatgttcaagagagaactatttccttcaaagacgatggaacttacaaaaccagagctgaagtcaaattcgagggtgatacattggttaacagaattgaattgaaaggaatcgatttcaaggaagatgggaatatccttggtcacaaattggaatacaatttcaattctcacaatgtgtatatcaccgccgacaagcagaaaaacggcattaaggctaactttaagataaggcacaatgtagaggatggatcagtgcaactagccgatcattatcaacagaatacacctattggagatgggcctgtacttttaccagataatcactacttatctacccaatctgttctgtcaaaggaccctaacgagaaaagagatcatatggtgttgttagaatttgtaacagcagcaggcataacacatggaatggacgaactttacaag

SEQ ID NO: 16 - DNA, 714 bp, Streptomyces coelicolor
atgagcaagggcgaagagctgttcacgggcgtcgtccccatcctggtggagctcgacggcgacgtcaacggccacaagttctccgtccgcggtgagggcgagggggacgccaccaacggcaagctgaccctcaagttcatctgcaccaccgggaagctgcccgtcccgtggccgaccctggtcaccaccctcacctacggcgtgcagtgcttctcccggtaccccgaccacatgaagcgccacgacttcttcaagtcggccatgccggaagggtacgtccaggagcggaccatcagcttcaaggacgacggtacgtacaagacgcgcgccgaggtgaagttcgaaggggacacgctggtgaaccggatcgagctgaagggcatcgacttcaaggaggacggcaacatcctggggcacaagctggagtacaacttcaactcgcacaacgtgtacatcaccgccgacaagcagaagaacggcatcaaggcgaacttcaagatccgccacaacgtcgaggacggctccgtccagctcgcggaccactaccagcagaacacgcccatcggcgacggtcccgtgctcctgccggacaaccactacctcagcacgcagtcggtgctgtccaaggacccgaacgagaagcgggaccacatggtgctgctcgagttcgtcaccgccgcgggcatcacccacggcatggacgagctgtacaag

SEQ ID NO: 17 - DNA, 714 bp, Trichoderma reesei
atgtccaagggcgaagagctgttcactggcgtcgtccccattctcgtggagctggacggcgacgttaatggccataagttctctgttcgcggagagggtgagggtgacgccacgaacggcaaacttaccctgaagtttatctgcaccaccggtaagcttcctgtgccgtggcccactctggtcaccacgctcacttacggagtgcagtgtttctcccgttaccccgaccacatgaagcgccatgatttcttcaagagcgctatgcctgagggttacgttcaggaacgaaccatcagcttcaaggatgacggaacatacaagactcgagctgaagtcaagttcgaaggtgacaccctcgtcaaccgtattgagttgaagggcatcgatttcaaagaggatggaaacatcctgggccacaagctcgagtacaactttaactcccacaatgtctacatcacggcggacaagcagaagaatggcattaaggccaactttaagattagacacaacgttgaggacggaagcgtccaactcgccgatcactatcagcaaaacacccccattggcgatggcccggttttgctcccagataaccactatctgtcgacacagtcagtcctctctaaggaccccaacgagaagcgcgatcacatggtccttcttgagttcgtgactgccgcaggaatcacccatggtatggacgagctgtacaaa

SEQ ID NO: 18 - DNA, 675 bp, Artificial Sequence
  DsRed2
atggcctcctccgagaacgtcatcaccgagttcatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcctgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctg

SEQ ID NO: 19 - DNA, 675 bp, Artificial Sequence
  DsRed2-mut
atggcctcctccgagaacgtcatcaccgagtacatgcgcttcaaggtgcgcatggagggcaccgtgaacggccacgagttcgagatcgagggcgagggcgagggccgcccctacgagggccacaacaccgtgaagctgaaggtgaccaagggcggccccctgcccttcgcctgggacatcctgtccccccagttccagtacggctccaaggtgtacgtgaagcaccccgccgacatccccgactacaagaagctgtccttccccgagggcttcaagtgggagcgcgtgatgaacttcgaggacggcggcgtggcgaccgtgacccaggactcctccctgcaggacggctgcttcatctacaaggtgaagttcatcggcgtgaacttcccctccgacggccccgtgatgcagaagaagacgatgggctgggaggcctccaccgagcgcttgtacccccgcgacggcgtgctgaagggcgagacccacaaggccctgaagctgaaggacggcggccactacctggtggagttcaagtccatctacatggccaagaagcccgtgcagctgcccggctactactacgtggacgccaagctggacatcacctcccacaacgaggactacaccatcgtggagcagtacgagcgcaccgagggccgccaccacctgttcctg

SEQ ID NO: 20 - DNA, 1542 bp, Artificial Sequence
  clone 3a3
aaattgaagagtttgatcatggctcagattgaacgtggcggcaggcctaacacatgcaagtcgagcggcagcggaaagtagcttgctactttgccggcgagcggcggacgggtgagtaatgtctgggaaactgcctgatggagggggataactactggaaacggtagctaataccgcatgacctcgcaagagcaaagtgggggaccttcgggcctcacgccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaagggtatcgtgttaatagcacggtacattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttagcagagatgctttagtgccttagggaaccgtgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 21 - DNA, 1541 bp, Artificial Sequence
  clone 2d11
aaattgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgataaggttaataaccttatcgattgacgttacccgcagaagaagcaccggctaactccgtgccagcagccgcggtaatacggagggtgcaagcgttaatcggaattactgggcgtaaagcgcacgcaggcggtctgtcaagtcagatgtgaaatccccgggcttaacctgggaactgcatttgaaactggcaggctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcgagtaatgtcgggaactcaaaggagactgccggtgacaaaccggaggaaggtggggatgacgtcaagtcatcatggcccttacggccagggctacacacgtgctacaatggcgcatacaaagagaagcgaactcgcgagagcaagcggacctcacaaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 22 - DNA, 1542 bp, Artificial Sequence
  clone 2ee4
aaattgaagagtttgatcatggctcagattgaacgctggcggcaggcctaacacatgcaagtcgagcggcagcggaaagtagcttgctactttgccggcgagcggcggacgggtgagtaatgtctgggaaactgcctgatggagggggataactactggaaacggtagctaataccgcatgacctcgcaagagcaaagtgggggaccttcgggcctcacgccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaagggtatcgtgttaatagcacggtacattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgctggggagtacggccgcaaggttaaaactcaaatgaattgacgggggcccgcacaagcggtggagcatgtggtttaattcgatgcaacgcgaagaaccttacctactcttgacatccacggaatttggcagagatgccttagtgccttcgggaaccgtgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccgggtaggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 23 - DNA, 1542 bp, Artificial Sequence
  clone 2g4
aaattgaagagtttgatcatggctcagattgaacgaggcggcaggcctaacacatgcaagtcgagcggcagcggaaagtagcttgctactttgccggcgagcggcggacgggtgagtaatgtctgggaaactgcctgatggagggggataactactggaaacggtagctaataccgcatgacctcgcaagagcaaagtgggggaccttcgggcctcacgccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaagggtatcgtgttaatagcacggtgcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttggcagagatgccttagtgccttcgggaaccgtgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 24 - DNA, 1543 bp, Artificial Sequence
  clone 2b10
aaattgaagagtttgatcatggctcagattgaacgctggcggcaggcctaacacatgcaagtcgagcggcagcggaaagtagcttgctactttgccggcgagcggcggacgggtgagtaatgtctgggaaactgcctgatggagggggataactactggaaacggtagctaataccgcatgacctcgcaagagcaaagtgggggaccttcgggcctcacgccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtgtgaagaaggccttcgggttgtaaagcactttcagcgaggaggaagggtatcgtgttaatagcacggtgcattgacgttactcgcagaagaagcaccggctaactccgtgccagcagccgcggtaatacggagggtgcaagcgttaatcggaattactgggcgtaaagcgcacgcaggcggtttgttaagtcagatgtgaaatccccgagcttaacttgggaactgcatttgaaactggcaagctagagtcttgtagaggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgctgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctactcttgacatccacggaatttggcagagatgccttagtgccttcgggaaccgtgagacaggtgctgcatggctgtcgtcagctcgtgttgtgaaatgttgggttaagtcccgcaacgagcgcaacccttatcctttgttgccagcgagtaatgtcgggaactcaaaggagactgccggtgataaaccggaggaaggtggggatgacgtcaagtcatcatggcccttacgagtagggctacacacgtgctacaatggcatatacaaagagaagcgaactcgcgagagcaagcggacctcataaagtatgtcgtagtccggattggagtctgcaactcgactccatgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 25 - DNA, 1542 bp, Artificial Sequence
  clone 1h12
aaattgaagagtttgatcatggctcagattgaacgctggcggcaggcctaacacatgcaagtcgaacggtagcacagagagcttgctctcgggtgacgagtggcggacgggtgagtaatgtctgggaaactgcctgatggagggggataactactggaaacggtagctaataccgcataacgtcttcggaccaaagtgggggaccttcgggcctcacaccatcggatgtgcccagatgggattagctagtaggtggggtaatggctcacctaggcgacgatccctagctggtctgagaggatgaccagccacactggaactgagacacggtccagactcctacgggaggcagcagtggggaatattgcacaatgggcgcaagcctgatgcagccatgccgcgtgtatgaagaaggccttcgggttgtaaagtactttcagcggggaggaaggcgataaggttaataaccttatcgattgacgttacccgcagaagaagcaccggctaactccgtgccagcagccgcggtaatacggagggtgcaagcgttaatcggaattactgggcgtaaagcgcacgcaggcggtctgtcaagtcagatgtgaaatccccgggcttaacctgggaactgcatttgaaactggcaggctagagtcttgtagagggggggtagaattccaggtgtagcggtgaaatgcgtagagatctggaggaataccggtggcgaaggcggccccctggacaaagactgacgctcaggtgcgaaagcgtggggagcaaacaggattagataccctggtagtccacgccgtaaacgatgtcgacttggaggttgtgcccttgaggcgtggcttccggagctaacgcgttaagtcgaccgcctggggagtacggccgcaaggttaaaactcaaatgaattgacgggggcccgcacaagcggtggagcatgtggtttaattcgatgcaacgcgaagaaccttacctggccttgacatccacggaattcggcagagatgccttagtgccttcgggaaccgtgagacaggtgctgcatggctgtcgtcagctcgtgttgtgaaatgttgggttaagtcccgcaacgagcgcaacccttatcctttgttgccagcgagtaatgtcgggaactcaaaggagactgccggtgacaaaccggaggaaggtggggatgacgtcaagtcatcatggcccttacggccagggctacacacgtgctacaatggcgcatacaaagagaagcgaactcgcgagagcaagcggacctcacaaagtgcgtcgtagtccggatcggagtctgcaactcgactccgtgaagtcggaatcgctagtaatcgtagatcagaatgctacggtgaatacgttcccgggccttgtacacaccgcccgtcacaccatgggagtgggttgcaaaagaagtaggtagcttaaccttcgggagggcgcttaccactttgtgattcatgactggggtgaagtcgtaacaaggtaaccgtaggggaacctgcggttggatcacctcctta

SEQ ID NO: 26 - DNA, 1543 bp, Artificial Sequence; identical to SEQ ID NO: 24
  clone 4g10

(19) United States
(12) Patent Application Publication
(10) Pub. No.: US 2013/0244904 A1
(43) Pub. Date: Sep. 19, 2013

(54) ESCHERICHIA COLI HAVING A MODIFIED TRANSLATIONAL PROPERTY

(71) Applicant: National Institute of Advanced Industrial Science and Technology, (US)

(72) Inventors: Kentaro Miyazaki, Sapporo-shi (JP); Miyuki Tsukuda, Sapporo-shi (JP)

(73) Assignee: National Institute of Advanced Industrial Science and Technology, Tokyo (JP)

(21) Appl. No.: 13/802,256

(22) Filed: Mar. 13, 2013

Related U.S. Application Data
(60) Provisional application No. 61/611,827, filed on Mar. 16, 2012.

(51) Int. Cl. C12N 15/70

(52) U.S. Cl. CPC C12N 15/70
USPC 506/10; 435/69.1; 506/14; 435/252.33

(57) ABSTRACT

By using Escherichia coli which comprises a 16S rRNA derived from a heterologous organisms, the expression efficiency of a variety of genes which were difficult to be expressed in Escherichia coli, including those from unknown microorganisms, can be improved.

This application claims priority under 35 U.S.C. §119 to U.S. Provisional Patent Application No. 61/611,827, filed on Mar. 16, 2012, which is incorporated in its entirety by reference.

### TECHNICAL FIELD

The present invention relates to a mutant strain of Escherichia coli which has an excellent ability to heterologously express a gene encoding a protein, a creation method thereof, a method for expressing a protein using the Escherichia coli strain and a protein produced thereby.

### BACKGROUND ART

Since gene cloning technology was developed, a technique, which comprises isolating from various organisms a gene encoding a protein, and expressing it using a heterologous organism as a host, has been used. As the host organism, not only Escherichia coli but also a variety of microorganisms including actinomycetes, yeasts, filamentous fungi and so on have been used. Among them, Escherichia coli is the most frequently used microorganism not only as a host for research purposes but also as a host for industrial purposes. As a conventional technique for optimizing the protein expression by Escherichia coli, techniques, which comprises increasing the transcriptional activity by utilizing an RNA polymerase gene intrinsic to Escherichia coli (Proc Natl Acad Sci USA 80: 4432-4436, 1983; and Gene 29: 251-254, 1984) or an RNA polymerase gene derived from a phage (J Mol Biol 189: 113-130, 1986; and Gene 56: 125-135, 1987), and/or controlling the promoter activity by adding an inducer or the like, to study optimum expression conditions, have been generally well known. Many cases where the efficiency of protein expression was dramatically improved by using these methods have been reported. However, despite many trials and errors, cases where the protein of interest was not produced at all and cases where, if the protein of interest was produced, the produced amount was small, have also been often reported. What kind of protein or nucleotide sequence is difficult to be expressed remains unknown; but it has been well known that the protein expression will be difficult when the relationship between the organism from which the gene to be expressed is derived and the host organism where the gene is expressed is phylogenetically distant (Curr Opin Biotechnol 20: 616-622, 2009).

### SUMMARY OF THE INVENTION

An object of the present invention is to create, for improvement of the protein expression efficiency, a host Escherichia coli which is capable of efficiently expressing genes derived from a wide variety of biological species.

In order to accomplish said object, the present inventors focused on ribosomes, which play the translation function in cells.

As attempts to improve the protein production ability by modifying a ribosome(s), a method in which a modified type of S12, one of ribosomal proteins, is used (Japanese Patent No. 4441170), a method in which a modified type of L11 is used (Japanese Unexamined Patent Application Publication No. 2007-300858) and the like have been known with regard to Escherichia coli. However, the attempt to improve the protein expression ability by modifying a ribosomal RNA(s) has not been reported at all. This is because, as is apparent also from the fact that 16S rRNAs have been used for the phylogenetic classification of species, 16S rRNAs are highly conserved between species, and have been believed to be difficult to be replaced with those derived from heterologous organisms. Replacements of 16S rRNAs between species as academic researches have been reported (Proc Natl Acad Sci USA 96: 1971-1976, 1999), but the analysis in terms of influences of a mutant ribosome(s) on the translation property and/or the protein expression has not been reported at all.

Under these circumstances, the present inventors focused on 16S rRNAs, which occupy the core of ribosomes and complexly interact with ribosomal proteins, and aimed to greatly change the function of the ribosomes. Consequently, the present inventors reconstructed in a host Escherichia coli cell ribosomal proteins carried by a host Escherichia coli with a 16S rRNA derived from a heterologous organism; changed the ability of heterologous expression by using the thus obtained hybrid type ribosomes; discovered that mutant strains of Escherichia coli in which expression of a gene of interest is enhanced are contained among them; and thereby completed the invention.

It is an aspect of the present invention to provide a method for expressing a protein, comprising introducing a gene of interest into a mutant strain of Escherichia coli which comprises a 16S rRNA gene derived from a heterologous organism, and expressing the protein encoded by the gene of interest.

It is another aspect of the present invention to provide the method as described above, wherein the 16S rRNA gene is derived from a metagenome.

It is another aspect of the present invention to provide the method as described above, wherein the 16S rRNA gene is derived from a proteobacterium.

It is another aspect of the present invention to provide the method as described above, wherein the proteobacterium is derived from a γ-proteobacterium.

It is another aspect of the present invention to provide the method as described above, wherein the proteobacterium is derived from a β-proteobacterium.

It is another aspect of the present invention to provide the method as described above, wherein the γ-proteobacterium belongs to the genus Serratia.

It is another aspect of the present invention to provide the method as described above, wherein the γ-proteobacterium is Serratia ficaria.

It is another aspect of the present invention to provide the method as described above, wherein the β-proteobacterium belongs to the genus Ralstonia, the genus Caldimonas, the genus Hydrogenophaga, the genus Oligella, the genus Oxalicibacterium, the genus Spirillum or the genus Burkholderia.

It is another aspect of the present invention to provide the method as described above, wherein the β-proteobacterium is Ralstonia pickettii, Caldimonas manganoxidans, Hydrogenophaga flava, Oligella urethralis, Oxalicibacterium horti, Spirillum lunatum or Burkholderia sacchari.

It is another aspect of the present invention to provide the method as described above, wherein the 16S rRNA gene derived from a metagenome has a sequence selected from the group consisting of SEQ ID NOs:20 to 26.

It is another aspect of the present invention to provide a library which comprises one or more mutant strain(s) of Escherichia coli which comprises a 16S rRNA gene derived from a heterologous organism.

It is another aspect of the present invention to provide a method of screening a host Escherichia coli strain in which a gene of interest is highly expressed, which comprises the steps of: introducing the gene of interest into the library as described above; and determining the expression amount of the gene of interest.

It is another aspect of the present invention to provide a host Escherichia coli strain in which a gene of interest is highly expressed, which strain is obtainable by the method as described above.

By using an Escherichia coli host strain comprising a 16S rRNA-modified type ribosome, a gene whose expression level is low in a wild type strain of Escherichia coli whose 16S rRNA has not been modified will be able to be highly expressed.

### BRIEF DESCRIPTION OF THE DRAWINGS

FIG. 1 shows the fluorescence intensity of each green fluorescent protein in the individual hosts.

FIG. 2 shows the comparison of GFP/Abi expression in the individual hosts. The ordinate represents the relative fluorescence intensity. The abscissa represents the names of the clones.

FIG. 3 shows the comparison of GFP/Bsu expression in the individual hosts. The ordinate represents the relative fluorescence intensity. The abscissa represents the names of the clones.

FIG. 4 shows the comparison of GFP/Eco expression in the individual hosts. The ordinate represents the relative fluorescence intensity. The abscissa represents the names of the clones.

FIG. 5 shows the comparison of GFP/Hsa expression in the individual hosts. The ordinate represents the relative fluorescence intensity. The abscissa represents the names of the clones.

FIG. 6 shows the comparison of GFP/Sce expression in the individual hosts. The ordinate represents the relative fluorescence intensity. The abscissa represents the names of the clones.

FIG. 7 shows the comparison of GFP/Sco expression in the individual hosts. The ordinate represents the relative fluorescence intensity. The abscissa represents the names of the clones.

FIG. 8 shows the comparison of GFP/Tre expression in the individual hosts. The ordinate represents the relative fluorescence intensity. The abscissa represents the names of the clones.

FIG. 9 shows the screening of the fluorescence from DsRed2 in the host library. The ordinate represents the relative fluorescence intensity. The abscissa represents the number of the clones.

FIG. 10 shows the comparison of DsRed2 expression. The ordinate represents the relative fluorescence intensity. The abscissa represents the names of the clones.

FIG. 11 shows the screening of the fluorescence from DsRed2-mut in the host library. The ordinate represents the relative fluorescence intensity. The abscissa represents the number of the clones.

FIG. 12 shows the comparison of DsRed2-mut expression. The ordinate represents the relative fluorescence intensity. The abscissa represents the names of the clones.

### DESCRIPTION OF THE EMBODIMENTS OF THE INVENTION

The term “16S rRNA gene derived from a heterologous organism” means a 16S rRNA gene which is derived from an organism other than Escherichia coli, preferably a bacterium other than Escherichia coli, more preferably a proteobacterium other than Escherichia coli. Examples of heterologous organisms include bacteria belonging to the genus Serratia, bacteria belonging to the genus Ralstonia, bacteria belonging to the genus Caldimonas, bacteria belonging to the genus Hydrogenophaga, bacteria belonging to the genus Oligella, bacteria belonging to the genus Oxahcibacterium, bacteria belonging to the genus Spirillum, bacteria belonging to the genus Burkholderia and the like.

The 16S rRNA genes derived from heterologous organisms may be obtained, for example, by carrying out PCR using as a template genomic DNA derived from a heterologous organism and using the pair of primers of SEQ ID NOs:1 and 2.

Alternatively, they may also be obtained, from genomic DNA derived from heterologous organism, by hybridization using as a probe a 16S rRNA gene derived from Escherichia coli.

As the mutant strain of Escherichia coli, one in which an endogenous 16S rRNA gene has been disrupted and, instead, a 16S rRNA gene derived from a heterologous organism has been introduced thereto is preferable.

The method for expressing a protein preferably comprises the steps of: introducing a gene encoding a protein of interest into a mutant strain of Escherichia coli which comprises a 16S rRNA gene derived from a heterologous organism; and expressing the protein of interest in the mutant strain.

The kind of the protein is not limited, but examples thereof include enzymes, cytokines, transcription factors, protein libraries and so on.

As the Escherichia coli to be used for the expression, Escherichia coli cells may be used as they are, or only a ribosomal fraction thereof may be used.

The screening of a host strain of Escherichia coli in which a gene of interest is highly expressed may be carried out by: introducing, into Escherichia coli in which an endogenous 16S rRNA gene has been disrupted, a library of 16S rRNA genes derived from heterologous organisms; introducing a gene encoding a protein of interest into the obtained library of mutant strains of Escherichia coli; expressing the protein of interest; and selecting a mutant strain in which the protein of interest is highly expressed. For example, the mutant strain in which a protein of interest is highly expressed may be selected as a strain in which the protein of interest is expressed in an amount more than the expression amount of the protein of interest in an Escherichia coli wild-type strain. Plasmids and so on which are widely used for Escherichia coli may be used for the gene introduction.

The growth of the Escherichia coli whose endogenous 16S rRNA gene has been disrupted is sometimes decreased, thus, when a library of 16S rRNA genes derived from heterologous organisms is introduced thereto, it is preferable that the Escherichia coli whose endogenous 16S rRNA gene has been disrupted be made to harbour the same 16S rRNA gene on a curable plasmid or the like, and a 16S rRNA gene derived from a heterologous organism be introduced thereinto at the same time as the elimination of the 16S rRNA gene on the plasmid. For example, the 16S rRNA gene can be eliminated by a method which comprises making the 16S rRNA gene to be harboured on a plasmid carrying sacB gene, and culturing in a medium containing sucrose.

By identifying the introduced 16S rRNA gene in the selected mutant strain, a 16S rRNA gene suitable for the expression of the protein of interest can be selected.

The term “metagenome” means a DNA library which is obtained by directly extracting DNAs from an environmental sample without isolating and culturing the microorganisms. The 16S rRNA genes derived from a metagenome (a library) contain(s) 16S rRNA genes from various microorganisms and may be obtained, for example, by carrying out PCR using as templates DNAs from an environmental sample and using the pair of primers of SEQ ID NOs:1 and 2.

### EXAMPLES

Examples of the present invention will now be described. However, the present invention is not limited thereto.

### Example 1

### Creation of Escherichia coli Strains Comprising 16S rRNAs Derived from Heterologous Microorganisms

(1) Preparation of 16S rRNA Genes from Isolated Microorganisms

As sources of 16S rRNA genes, the following bacterial strains obtained from National Institute of Technology and Evaluation were used. The descriptions in the parentheses are reference numbers of the bacterial strains. Individual 16S rRNA genes from Serratia ficaria (NBRC 102596), Ralstonia pickettii (NBRC 102503), Caldimonas manganoxidans (NBRC 16448), Hydrogenophaga flava (NBRC 102514), Oligella urethralis (NBRC 14589), Oxalicibacterium horti (NBRC 13594), Spirillum lunatum (NBRC 13958), Burkholderia sacchari possessed by the inventors' laboratory and Escherichia coli MG1655 possessed by inventors' laboratory were amplified by PCR.

For the amplification of the 16S rRNA gene fragments, the oligonucleotides represented by SEQ ID NOs:1 and 2 (these are both manufactured by Hokkaido System Science Co., Ltd.) were used as primers. Regions corresponding to almost full length of the 16S rRNA genes were amplified by a polymerase chain reaction (PCR) method using as templates the chromosomal DNAs derived from the individual microorganisms. Each reaction solution (total amount: 25 μL) contains 1×PCR Buffer for KOD FX Neo (manufactured by TOYOBO), 0.2 mM each of dATP, dGTP, dCTP and dTTP (these are all manufactured by TOYOBO), 25 pmol each of the primers, 1 μL of the genomic DNA (about 30 ng) and 0.5 units of KOD FX Neo (manufactured by TOYOBO). For these solutions, incubation was performed at 98° C. for 2 minutes; thereafter a temperature cycle of 98° C. for 10 seconds, 55° C. for seconds and 68° C. for 45 seconds was repeated 25 times; and then finally incubation at 68° C. for 5 minutes was performed. The obtained PCR products were separated by agarose gel electrophoresis, purified by using a DNA purification kit manufactured by MACHEREY-NAGEL (NucleoSpin Extract II) according to the manual, and eluted with 30 μL of water.

(2) Preparation of 16S rRNA Genes Derived from Metagenome from Environmental Sample

Environmental genomic DNA was prepared from the soil collected at Toyohira-ku, Sapporo, Hokkaido, Japan. About 500 ng (100 μL) of genomic DNA was prepared from 1 g of the soil sample by using Extrap Soil DNA Plus ver. 2 manufactured by J-Bio 21 according to the manual of the kit.

For the amplification of the 16S rRNA genes, the oligonucleotides represented by SEQ ID NOs:1 and 2 were used as primers. Regions corresponding to almost full length of the 16S rRNA genes were amplified by a PCR method using as templates the genomic DNA prepared from the soil sample. The PCR conditions are as follows. Each reaction solution (Total Amount: 25 μL) contains a final concentration of 1×PCR Buffer for KOD FX Neo (manufactured by TOYOBO), 0.2 mM each of dATP, dGTP, dCTP and dTTP (these are all manufactured by TOYOBO), 25 pmol each of the primers, 1 μL of the genomic DNAs (about 30 ng) and 0.5 units of KOD FX Neo (manufactured by TOYOBO). For these solutions, incubation was performed at 98° C. for 2 minutes; thereafter a temperature cycle of 98° C. for 10 seconds, 55° C. for 30 seconds and 68° C. for 45 seconds was repeated 25 times; and then finally incubation at 68° C. for 5 minutes was performed. The obtained PCR products were separated by agarose gel electrophoresis according to a conventional method, purified by using a DNA purification kit manufactured by MACHEREY-NAGEL (NucleoSpin Extract II) according to the manual, and eluted with 30 μL of water.

(3) Cloning of 16S rRNA Genes Derived from Heterologous Microorganisms and Construction of Expression System

By the Megawhop method (Biotechniques. 33(5): 1033-4, 1036-8, 2002), the 16S rRNA gene fragments obtained in (1) and (2) above were used to replace the 16S rRNA derived from Escherichia coli on plasmid pRB 103 containing the Escherichia coli rrnB operon as described in Mol. Cell. 34: 760-766, 2009. Each reaction solution (Total Amount: 25 μL) contains 1×PCR Buffer for KOD Neo (manufactured by TOYOBO), 0.2 mM each of dATP, dGTP, dCTP and dTTP (these are all manufactured by TOYOBO), 25 pmol each of the primers, 5 μL of the 16S rRNA gene fragments obtained in (1) and (2) above and 0.5 units of KOD Neo (manufactured by TOYOBO). For these solutions, incubation was performed at 68° C. for 5 minutes, followed by incubation at 98° C. for 2 minutes; thereafter a temperature cycle of 98° C. for 10 seconds, 55° C. for 30 seconds and 68° C. for 45 seconds was repeated 18 times; and then finally incubation at 68° C. for 5 minutes was performed. To each of these solutions after the reaction, 0.5 μL of a restriction enzyme DpnI (manufactured by NEB) was added, and the mixture was incubated at 37° C. overnight. Thereafter, purification was carried out by using a DNA purification kit manufactured by MACHEREY-NAGEL (NucleoSpin Extract II), and elution with 30 μL of water was carried out. An aliquot of the each solution was taken, and used to transform the Escherichia coli KT101 rna⁻ strain as described in Mol. Cell. 34: 760-766, 2009 (a strain in which all of the seven rrn operons on the Escherichia coli chromosome have been deleted, but which harbors the pRB101 containing the rrnB operon).

(4) Creation of Host Strains Comprising 16S rRNA Genes Derived from Heterologous Species

In the Escherichia coli KT101 rna⁻ strain, all rrn operons on the chromosome have been deleted, but the growth is compensated by the Escherichia coli rrnB operon encoded by the pRB101. The pRB101 also encodes the sacB gene derived from Bacillus subtilis, and the plasmid can be eliminated by performing selection in a medium containing sucrose. Accordingly, if the selection of the Escherichia coli transformed in the step (3) above is carried out based on the growth compensation thereof in the presence of sucrose, then selection of 16S rRNA genes functioning in the Escherichia coli will be enabled.

Experimental procedures for this are as follows.

<Creation of Mutant Strains which Comprise Plasmids pRB103 Comprising 16S rRNA Gene Derived from Isolated Bacterial Strains>

First, the transformants were inoculated on an LB Lennox agar medium containing 100 μg/mL Zeocin (manufactured by Invitrogen) (the LB medium and the agar are both manufactured by Merck). Next, the colonies appeared on the plates were picked up using pipette tips, and suspended in 100 μL of LB medium, and thereafter again inoculated on an LB agar medium containing 100 μg/mL Zeocin and 5% sucrose (manufactured by Wako Pure Chemical Industries, Ltd.). After culturing at 37° C. overnight, selection of grown colonies was carried out. As a result, the growth of individual colonies comprising the 16S rRNA gene derived from Serratia ficaria (SEQ ID NO:3), the 16S rRNA gene derived from Ralstonia pickettii (SEQ ID NO:4), the 16S rRNA gene derived from Caldimonas manganoxidans (SEQ ID NO:5), the 16S rRNA gene derived from Hydrogenophaga flava (SEQ ID NO:6), the 16S rRNA gene derived from Oligella urethralis (SEQ ID NO:7), the 16S rRNA gene derived from Oxalicibacterium horti (SEQ ID NO:8), the 16S rRNA gene derived from Spirillum lunatum (SEQ ID NO:9) and the 16S rRNA gene derived from Burkholderia sacchari (SEQ ID NO:10) was confirmed. The thus obtained modified hosts were subjected to the following protein expression experiment. In particular, the mutant strain comprising the 16S rRNA gene derived from Serratia ficaria was designated KT103 rna⁻/Sfi; the mutant strain comprising the 16S rRNA gene derived from Ralstonia pickettii was designated KT103 rna⁻/Rpi; the mutant strain comprising the 16S rRNA gene derived from Caldimonas manganoxidans was designated KT103 rna⁻/Cma; the mutant strain comprising the 16S rRNA gene derived from Hydrogenophaga flava was designated KT103/Hfl; the mutant strain comprising the 16S rRNA gene derived from Oligella urethralis was designated KT103 rna⁻/Our; the mutant strain comprising the 16S rRNA gene derived from Oxalicibacteriurn horti was designated KT103 rna⁻/Oho; the mutant strain comprising the 16S rRNA gene derived from Spirillum lunatum was designated KT103 rna⁻/Slu; the mutant strain comprising the 16S rRNA gene derived from Burkholderia sacchari was designated KT103 rna⁻/Bsa; and the host strain comprising the 16S rRNA gene derived from Escherichia coli rrnB was designated KT103 rna⁻/Eco. In respect to the nucleotide sequences of SEQ ID NOs:3 to 10 and SEQ ID NOs:20 to 26 mentioned below, the gene sequence of the region between the amplification primers for the 16S rRNA genes and the sequence outside the amplified region including the amplification primers are represented in combination with the homologous region of the 16S rRNA derived from Escherichia coli.

<Creation of Mutant Strains which Comprise Plasmid pRB103 Comprising 16S rRNA Gene Derived from Metagenome>

In creating the mutant strains which harbour the 16S rRNA gene obtained from the environmental microorganisms, colonies which grew on an LB Lennox agar medium containing 100 μg/mL Zeocin were suspended in 3 mL of LB medium, and the resultant was stirred by a vortex mixer for 1 minute. An aliquot thereof was again inoculated on an LB agar medium containing 100 μg/mL Zeocin and 5% sucrose.

### Example 2

### Protein Expression by Hosts Comprising 16S rRNA Gene Derived from Isolated Strain

### (1) Transformation of Mutant Host Strains

Using the host strains created in Example 1 which has been introduced with the 16S rRNA gene derived from the isolated bacterial strain, competent cells were produced according to the procedure shown in Sambrook et al., Molecular Cloning: A Laboratory Manual third edition. Cold Spring Harbor Laboratory Press. 1.105. 2001. These cells were transformed with plasmid expressing the seven green fluorescent proteins described below. The expression plasmids for the green fluorescent proteins were obtained by cloning each of the genes encoding the green fluorescent proteins into the plasmid pJexpress402 provided by DNA2.0 (containing a chloramphenicol resistant marker). The cloned green fluorescent proteins have the same amino acid sequence, but the nucleotide sequences thereof are different from each other. The green fluorescent proteins are proteins derived from Aequorea victoria, but the individual synthesized genes of the seven proteins were designed to suit the gene properties of seven biological species phylogenetically different from each other, i.e., Agaricus bisporus, Bacillus subtilis, Escherichia coli, Homo sapiens, Saccharomyces cerevisiae, Streptomyces coelicolor and Trichoderma reesei. The algorithm for the gene synthesis was by the DNA2.0's method. Hereinafter, these green fluorescent proteins are respectively abbreviated as GFP/Abi, GFP/Bsu, GFP/Eco, GFP/Hsa, GFP/Sce, GFP/Sco and GFP/Tre. SEQ ID NOs:11 to 17 respectively represent the nucleotide sequences of these green fluorescent proteins. As a control wild type Escherichia coli whose 16S rRNA is not modified, MG1655 (our laboratory's property) was used.

(2) Cultivation of Transformants and Evaluation of Fluorescence from Green Fluorescent Proteins

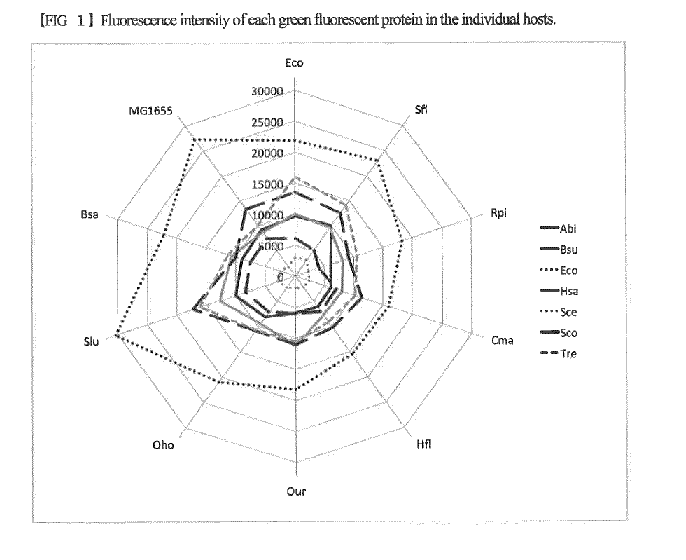

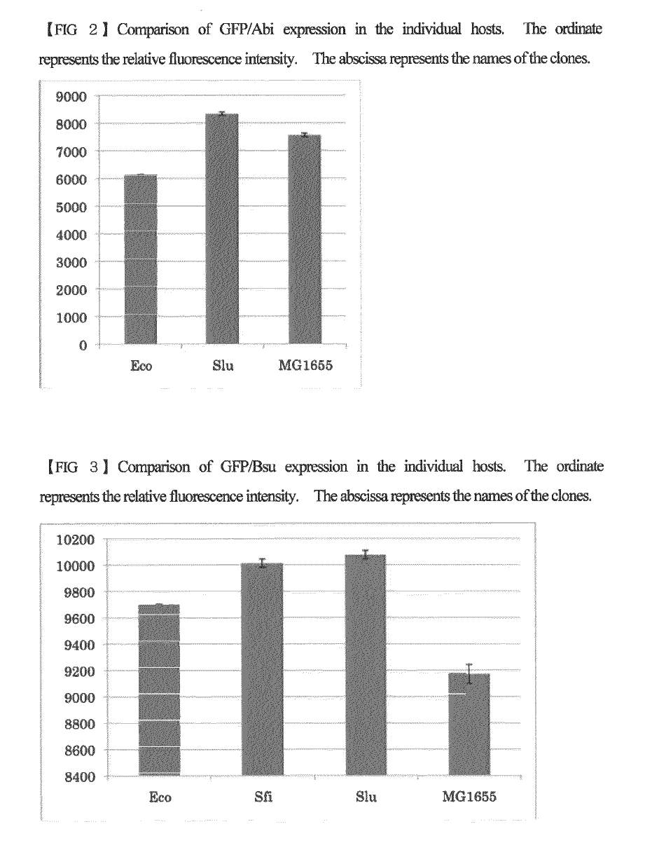

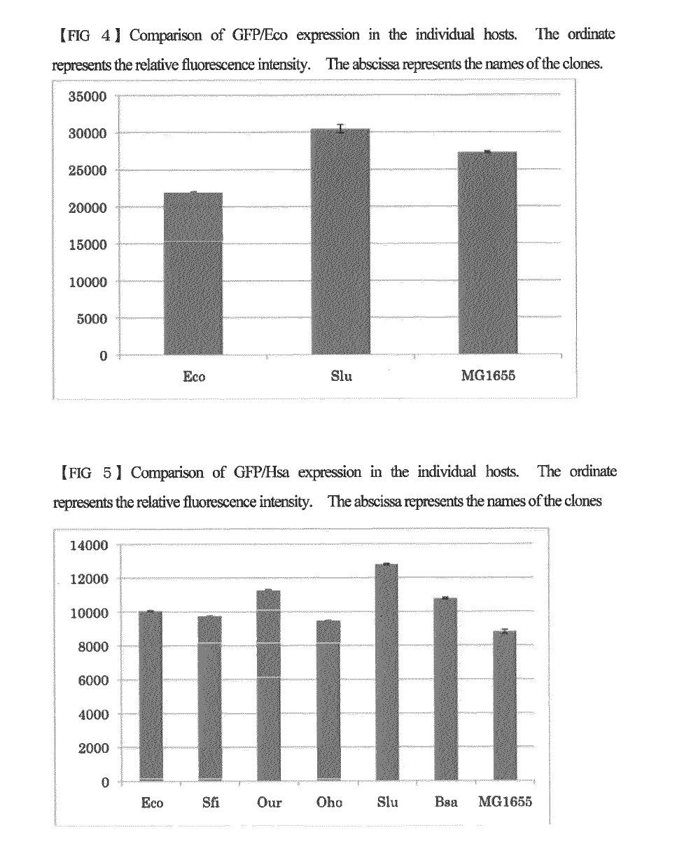

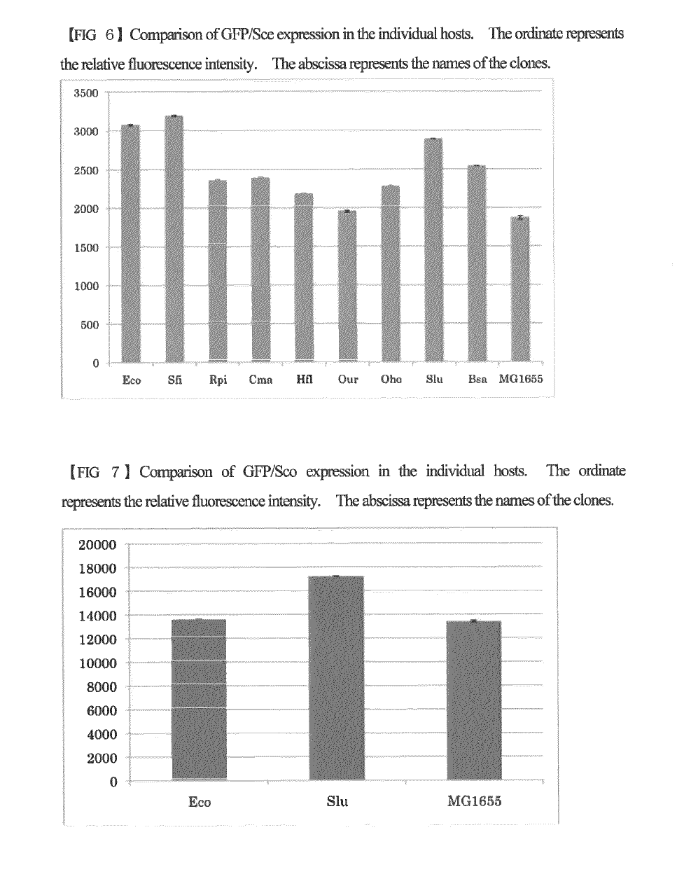

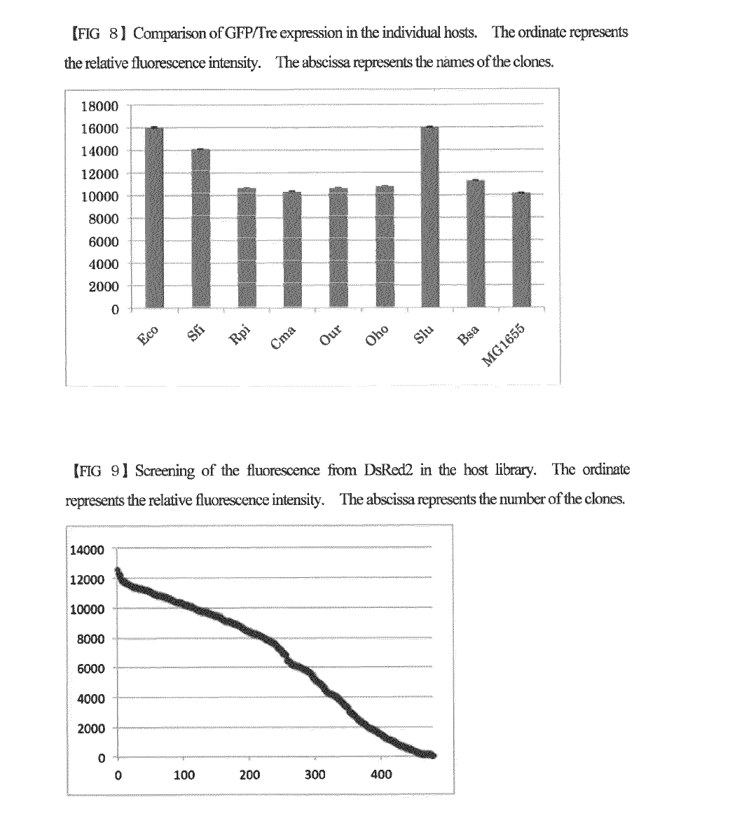

The transformants of the green fluorescent protein genes obtained in (1) above were cultured at 37° C. overnight in 1 mL of 2×YT medium containing 34 μg/mL chloramphenicol (N=8). As the culture vessel, a 96-well deep well plate manufactured by Greiner was used, and, as the shaking incubator, M•BR-024 manufactured by Taitec was used. Into 1 mL of 2×YT medium containing 34 μg/mL chloramphenicol and 0.1 mM isopropyl-β-thiogalactoside, 1 μL of the bacterial cells cultured overnight was innoculated, followed by culturing at 37° C. for 20 hours. An aliquot of the each culture (100 μL) was taken, and the fluorescence from the bacterial cells was measured in a black-bottom, 96-shallow well microplate at an excitation wavelength of 488 nm and a fluorescence wavelength of 530 nm. For the measurement, Gemini XS, a plate reader manufactured by Molecular Devices, was used. FIG. 1 shows the fluorescence intensity of each green fluorescent protein in the individual hosts. From the result, it was found that the protein expression efficiency is different between the hosts, and the hosts have expression specificities to genes having different properties. FIGS. 2 to 8 are graphs showing the comparison of KT103 rna⁻/Eco with those in which the expression of each green fluorescent protein is higher than that of MG1655 (a wild type). In these figures, KT103 rna⁻/Eco or the like is represented simply as Eco or the like, and other biological species and clones are also represented simply in the same manner.

As is apparent from these figures, the combinations of hosts and reporter genes in which the expression efficiency is higher than that in MG1655 (a wild-type strain) were observed. The combinations were as follows. That is, expression efficiencies higher than that in MG1655 were provided in: KT103 rna⁻/Slu with regard to GFP/Abi expression; KT103 rna⁻/Sfi and KT103 rna⁻/Slu with regard to GFP/Bsu; KT103 rna⁻/Slu with regard to GFP/Eco; KT103 rna⁻/Eco, KT103 rna⁻/Sfi, KT103 rna⁻/Our, KT103 rna⁻/Oho, KT103 rna⁻/Slu and KT103 rna⁻/Bsa with regard to GFP/Hsa; KT103 rna⁻/Sfi, KT103 rna⁻/Rpi, KT103 rna⁻/Cma, KT103 rna⁻¹/Hfl, KT103 rna⁻/Our, KT103 rna⁻/Oho, KT103 rna⁻/Slu and KT103 rna⁻/Bsa with regard to GFP/Sce; KT103 rna⁻/Slu with regard to GFP/Sco; and KT103 rna⁻/Eco, KT103 rna⁻/Sfi, KT103 rna⁻/Rpi, KT103 rna⁻/Cma, KT103 rna⁻/Our, KT103 rna⁻/Oho, KT103 rna⁻/Slu and KT103 rna⁻/Bsa with regard to GFP/Tre. From these results, it was proved that the expression efficiency of the wild type ribosome (KT103 rna⁻/Eco) varies depending on the genes, and that, with regard to some genes, the hybrid type ribosome which forms a complex with a 16S rRNA derived from a heterologous species exhibits an expression efficiency higher than that of the wild type Escherichia coli. As an especially remarkable example, it was found that KT103 rna⁻/Slu exhibits an expression efficiency higher than that in MG1655 with regard to all of these green fluorescent proteins.

In other words, it was proved that there exist strain, in which a gene that has been thought to be difficult to be expressed in a wild-type strain is efficiently expressed, among the mutant strains of Escherichia coli comprising heterologous 16S rRNA.

### Example 3

### Protein Expression by Host Library Comprising 16S rRNA Genes Derived from Metagenome

### (1) Transformation of Mutant Host Strain

Using the host library created in Example 1 which has been introduced with the 16S rRNA genes derived from the metagenome, competent cells were produced according to the procedure shown in Sambrook et al., Molecular Cloning: A Laboratory Manual third edition. Cold Spring Harbor Laboratory Press. 1.105. 2001. The competent cells were transformed with plasmid expressing red fluorescent proteins. As the red fluorescent proteins, DsRed2 manufactured by Clontech (SEQ ID NO:18) and DsRed2-mut, a mutant containing a double nucleotide substitution site (SEQ ID NO:19) were used.

(2-1) Evaluation of Fluorescence from Red Fluorescent Protein DsRed2

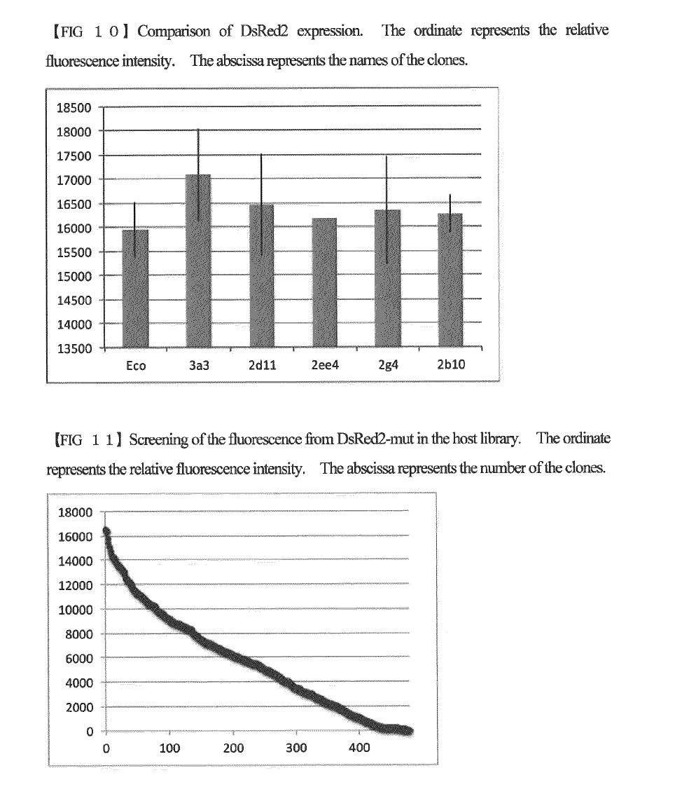

The 480 colonies of transformants of the red fluorescent protein genes obtained in (1) above were inoculated into 1 mL of LB medium containing 100 μg/mL ampicillin (a 96-well deep well plate manufactured by Greiner), and cultured under shaking at 37° C. using M•BR-024 manufactured by Taitec (N=8). Into 1 mL of LB medium (containing 100 μg/mL ampicillin and 0.1 mM isopropyl-β-thiogalactoside), 1 μL of the bacterial cells cultured overnight was innoculated, followed by culturing at 37° C. for 20 hours. Thereafter, the medium was removed by centrifugation (4000 rpm, for 5 minutes), and the precipitated bacterial cells were resuspended in 250 μL of sterile water. An aliquot of the bacterial suspension (100 μL) was taken, and the fluorescence from the bacterial cells was measured using Gemini XS, a plate reader manufactured by Molecular Devices, in a black-bottom, 96-well microplate manufactured by Nunc at an excitation wavelength of 554 nm and a fluorescence wavelength of 580 nm. The results are shown in FIG. 9. FIG. 9 is a graph obtained by sorting in descending order based on the fluorescence intensity of the clones. From the result, it was proved that the expression of the red fluorescent protein greatly varies depending on the mutant strains. This result means that the 16S rRNA-mutated type strains of Escherichia coli, which have been created by the method described in <Example 1> above, exhibit an expression property specific to the gene to be expressed. By constructing the mutant library, the gene difficult to be expressed in a wild type Escherichia coli can be efficiently expressed. In addition, several kinds of clones in which the fluorescence is higher than that of KT103 rna⁻/Eco (a control) were selected and remeasured. As a result, it was confirmed that clones which exhibit fluorescence higher than that of KT103 rna⁻/Eco were obtained. The fluorescence values of these clones and KT103 rna⁻/Eco were shown in FIG. 10. The names under the individual bars indicate the origins of the 16S rRNAs.

The gene sequences of the 16S rRNAs contained by these clones were shown in SEQ ID NOs:20 to 24.

(2-2) Evaluation of Fluorescence from Red Fluorescent Protein DsRed2-Mut

The transformant library obtained by introducing 16S rRNA genes from the metagenome was cultured and expression was measured in the same manner as in (2-1) above.

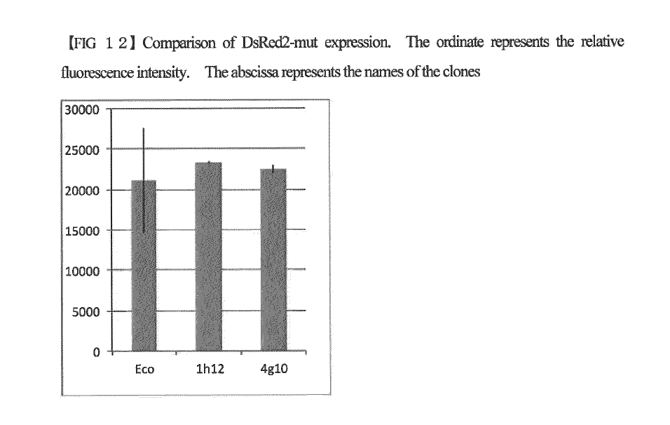

As a result, as shown in FIG. 11, it was proved that the expression of the red fluorescent protein greatly varies depending on the mutant strains. This result means that the 16S rRNA-mutated type strains of Escherichia coli, which have been created by the method described in <Example 1> above, exhibit an expression property specific to the gene to be expressed. By constructing the mutant library, the gene difficult to be expressed in a wild type Escherichia coli can be efficiently expressed.

In addition, several kinds of clones in which the fluorescence is higher than that of KT103 rna⁻/Eco (a control) were selected and remeasured. As a result, it was confirmed that clones which exhibit fluorescence higher than that of KT 103 rna⁻/Eco were obtained. The fluorescence values of these clones and KT103 rna⁻/Eco were shown in FIG. 12.

The gene sequences of the 16S rRNAs contained by these clones were shown in SEQ ID NOs:25 to 26.

Thus, by constructing hybrid ribosomes using a variety of 16S rRNAs, an expression level higher than that in Escherichia coli wild-type strain will be able to be achieved.

### INDUSTRIAL APPLICABILITY

By using as a host a mutant strain derived from Escherichia coli, a gene which has been considered to be difficult to be expressed in conventional wild type Escherichia coli will be able to be expressed. Accordingly, by a method which comprises, for example, constructing a metagenome library using such hosts, the efficiency of searching for a novel gene will be improved. In addition, enhancement of expression of various genes difficult to be expressed may contribute to a variety of fields, such as the food industry, the livestock industry and the energy industry.

While the invention has been described in detail with reference to preferred embodiments thereof, it will be apparent to one skilled in the art that various changes can be made, and equivalents employed, without departing from the scope of the invention. Each of the aforementioned documents, including the priority document, U.S. Provisional Patent Application No. 61/611,827, is incorporated by reference herein in its entirety.

1. An Escherichia coli mutant strain which comprises a 16S mRNA gene derived from a heterologous organism.

2. The Escherichia coli mutant strain according to claim 1, wherein the 16S mRNA gene is derived from a metagenome.

3. The Escherichia coli mutant strain according to claim 1, wherein the 16S rRNA gene is derived from a proteobacterium.

4. The Escherichia coli mutant strain according to claim 3, wherein the proteobacterium is derived from a γ-proteobacterium.

5. The Escherichia coli mutant strain according to claim 3, wherein the proteobacterium is derived from a β-proteobacterium.

6. The Escherichia coli mutant strain according to claim 4, wherein the γ-proteobacterium belongs to the genus Serratia.

7. The Escherichia coli mutant strain according to claim 4, wherein the γ-proteobacterium bacterium is Serratia ficaria.

8. The Escherichia coli mutant strain according to claim 5, wherein the β-proteobacterium belongs to the genus Ralstonia, the genus Caldimonas, the genus Hydrogenophaga, the genus Oligella, the genus Oxalicibacterium, the genus Spirillian or the genus Burkholderia.

9. The Escherichia coli mutant stain according to claim 5, wherein the β-proteobacterium is Ralstonia pickettii, Caldimonas manganoxidans, Hydrogenophaga flava, Oligella urethralis, Oxalicibacterium horti, Spirillum lunatum or Burkholderia sacchari.

10. The Escherichia coli mutant strain according to claim 2, wherein the 16S rRNA gene derived from a metagenome is any one or more of SEQ ID NOs:20 to 26.

11. A Library which comprises at least two Escherichia coli mutant strains according to any one of claims 1 to 10.

12. A method for screening a host Escherichia coli strain in which a gene of interest is highly expressed by using the mutant stain library according to claim 11.

13. A host Escherichia coli strain in which a gene of interest is highly expressed, which stain can be obtained by the method according to claim 12.

14. A method for expressing a protein by using the Escherichia coli mutant strain according to any one of claims 1 to 10.
